(19) United States
(12) Patent Application Publication
(10) Pub. No.: US 2009/0210030 A1
(43) Pub. Date: Aug. 20, 2009

(54) METHOD AND APPARATUS FOR IMPROVING NEUROMUSCULAR PERFORMANCES

(76) Inventor: Guido Maria Filippi, Rome (IT)

Correspondence Address: DICKSTEIN SHAPIRO LLP, 1177 AVENUE OF THE AMERICAS (6TH AVENUE), NEW YORK, NY, 10036-2714, US

(21) Appl. No.: 12/372,311

(22) Filed: Feb. 17, 2009

Related U.S. Application Data
(63) Continuation of application No. 10/498,943, filed on Nov. 24, 2004, now abandoned.

(30) Foreign Application Priority Data
Dec. 17, 2001 (IT) RM2001A000743

(51) Int. Cl. A61N 1/00

(52) U.S. Cl. 607/48

(57) ABSTRACT

The invention concerns a method for improving neuromuscular performances, characterised in that it is based on an Alpha-conditioning technique, carried out by a suitable stimulus of the nervous sensors during the voluntary muscular contraction, in that it transforms the mechanical properties of the soft tissues, developing a high-pass mechanical filter, in that it delivers a controlled and that can be modulated mechanical force signal destined to be read by force isometric nervous receptors, and in that it creates an illusory perception of the articular positioning.

The invention further relates to an apparatus for improving the neuromuscular performances, characterised in that it comprises at least a transducer, a transducer fixing system, and a control panel, said transducer having to support a static load adequate to transform biological tissues into a high-pass filter, imposing and sustaining a dynamic additive load on the muscular groups to be subjected to the treatment, thus producing a force signal able to propagate within the tissues, and being able to transmit said force signal to the patient; said control panel having to deliver an electric signal characterised by a pre-established frequency, being it possible to modulate the amplitude of said frequency (to reach the deep muscle and its articulation).

### CROSS-REFERENCE TO RELATED APPLICATIONS

This application is a continuation of U.S. application Ser. No. 10/498,943, filed Nov. 24, 2004, which is a National Stage Application of PCT/IT2002/000794, filed Dec. 16, 2002, which claims priority to Italian Application No. RM2001A000743, filed Dec. 17, 2001, the entirety of which is incorporated herein by reference.

The present invention relates to a method and apparatus for improving neuromuscular performances.

More specifically, the invention concerns a method, and an apparatus, of the above kind, based on an integrated alpha-conditioning and virtual realty creation action.

Still more specifically, the invention concerns a method and an apparatus able to transform the mechanical properties of the cutaneous, adipose and muscular soft tissues into mechanical properties of a high-pass mechanical filter. The invention allows to exploit the force isometric nervous sensors of the muscle-tendinous and osteo-articular groups in order to improve the motion performance using an alpha-conditioning paradigm, associated to the use of techniques specific of the virtual realty.

As it is well known, human motion nervous system uses, for its proper operation a continuous, incessant and enormous data collection, either data concerning the inside status and data about the outside realty. Said collection occurs by a continuous abstraction process from the outside and inside realty of a very large variety of parameters (mechanical, visual, biochemical, thermal, electromagnetic, etc.) (Kandel E R Eds Kandel E R, Schwartz J H and Jessell T M. Elsevier NY. pp 1009-1031. 1991).

Choose of said variables has been carried out since from the evolution on the basis of their relevance for surviving, i.e. on the basis of the outside and inside realty only some aspects are revealed, on the basis of which the realty image daily experimented is created.

Reading of checked variables controlled by the Central Nervous System has been carried out by a group of sensors (e.g. hundredths of thousands of sensors in a single muscle) specialised for reading of particular information (temperature, pressure, chemical molecules, length, force, torsion, light, etc.). Furthermore, each one of said specialisation is different inside for its dynamic features: some sensors are able to obtain the signal more than once with respect to the time, while others only read the static component.

On the basis of the information obtained, the Central Nervous System builds a model of the outside realty. Thanks to this model, the Central Nervous System takes its decisions, and in case the latters are repeated, it makes them becoming a motion program, behaving as a software. Further, Central Nervous System is able to modify its neuronal circulation (thus it is able to modify its hardware), making the same suitable to the new software (e.g. learning to ride a bike means elaborating a new software and a new and more suitable hardware).

Since, as already said, Central Nervous System abstracts from realty only some parameters and not all of them, to modify the perception of what is all about us, it is sufficient to provide to the Central Nervous System controlled variations only of some controlled parameters. Virtual realty tests are based on said verifications and are now used for the motion rehabilitation therapy (Rizzo A A, Buckwalter J G Stud Health Technol Inform 44: 123-145; 1997)).

Since Central Nervous System uses the informative system to make its motion programs, and possibly, to modify its hardware, it is consequently possible to provide to the Central Nervous System a given group of information to shortly obtain a stronger and more efficient reprogramming of the nervous circuits.

Biological tissues comprising the human body, particularly cutis, subcutis and muscles have such compliances to allow the transmission of low frequency mechanical signals (up to 20-30 Hz). Thus, they are low-pass structures, in case said compliances are saturated by compression, it is possible the transmission of mechanical signals with higher frequencies (up to 200 Hz). In fact, this procedure brings to the saturation of the elastic components and puts the viscous ones under pre-tensioning. Thus, the present invention is able to modify the mechanical properties of the biological tissues, transforming them into high-pass structures (assuming in the biological field that high frequencies are those in the order of hundredths of Hz).

It is also known that since from some decades it has been widely demonstrated that it is possible to introduce in animals (and in human being) some stereotyped behaviours following to the repeated presentation of suitable sequences of sensorial stimulus. Said behaviours have been indicated as “acquired reflexes” and procedures to induce them conditioning paradigms.

Said phenomenons have been, and are, widely studied, and their electrical, neural and molecular substrates progressively focused (Byrne J H Physiol Rev 67: 329-439. 1987)). In these studies (Wolpaw J R. J Neurophysiol 57: 443-458. 1987; Wolpaw J R. Med Sci Sports Exerc 16: 1475-1479. 1994) it has been demonstrated how some areas of the Central Nervous System can be modified if the cerebral area is susceptible of conditioning, if it is reached from suitable qualified stimulus, if it is reached from suitable intensity stimulus and if it is suitable their time distribution.

Furthermore, it has been put into evidence that said modifications are first of all biochemical, then involving structural macromolecules, thus determining permanent modifications (Mendell L M Physiol Rev 64: 260-324. 1984)).

Exemplifying the above, it can be said that conditioning paradigms determine modifications of the Central Nervous System software and hardware.

Conditioning paradigms individuated until today as useful to induce some modifications are numerous; some of them exploits simple methods, others require the help of complex instruments (it should be sufficient to think to the medical therapies based on the use of bio-feedbacks, neuronal conditioning therapies called operative). Common feature of these methods and apparatuses is the use of one or more sensitive ways to access the Central Nervous System and its circulation and the use of its exits (motion vie) to obtain the wished effects.

Said conditioning techniques, even being a potential and important therapeutic help (Wolpaw J R. Med Sci Sports Exerc 16: 1475-1479. 1994), since dry, does not require the use of drugs and obtain powerful and persistent effects. However, they have remarkable applicative difficulties, and particularly they require long applicative times (weeks or months). Furthermore, during the conditioning period it is required the diligent and continuous co-operation of the patient. Said co-operation is full and valid only for few subjects, aware of what they must do and having a medium-high cultural level. If the co-operation lacks, results are often absent or scarce, or completely transitory. Finally, it is in any case required the constant presence of an operator helping and following the conditioning process.

In the following, it will be taken into consideration the problem of configuring and apparatus and a method suitable to produce a neuronal conditioning destined to increase the motive performances and to use the muscle-articular isometric force sensors.

From the evaluations made during the years, a valid apparatus for the neural conditioning has two basic problems:
it must be able to produce a therapeutically valid neural conditioning;
it must be able to annul, or at least to minimise, the role of the co-operation required to the patient.

Particularly, first aspect requires that apparatus must have absolutely specific requirements, i.e. is must be able to use soft tissues and the muscle as interface between the same apparatus and the muscular, tendinous, articular and osteo-ligament sensorial system. All the above makes it deeply different from a simple muscular or cutaneous masseur, or from a circulation reactivating device.

Furthermore, particularly, it is necessary to be able to provide to said sensors a signal the values of which (frequency, amplitude, time duration, decaying features due to the soft tissues) are parameterised in a code (Byrne J H Physiol Rev 67: 329-439. 1987; Kandel E R Eds Kandel E R, Schwartz J H and Jessell T M. Elsevier NY. pp 1009-1031. 1991; Wolpaw J R. Med Sci Sports Exerc 16: 1475-1479. 1994)) suitable to produce a conditioning paradigm. Furthermore, said parameterisation will have to make an articular virtual realty representation to make spontaneous the patient co-operation (Roll J P, Vedel J P Exp Brain Res 47: 177-90; 1982; Sitting A C, Denier van der Gon J J, Gielen C C Exp Brain Res 67: 33-40; 1987; Verschueren S M, Cordo P J, Swinnen S P J Neurophysiol 79: 2265-76; 1998).

In the literature, a plurality of methods and/or apparatuses exists able to produce time variable forces (sinusoids, sawtooth, triangular, etc.). Among them, they can be mentioned the Italian Patent No. 1,277,959, and the U.S. Pat. Nos. 3,364,921, 3,984,708, 4,549,535, 4,895,149, 5,085,207, 5,101,810, 5,113,852, 5,279,284, 5,361,437, 5,437,608, 5,519,292, 5,611,771 and 5,780,958.

Among the solutions described in said documents, only that of the Italian Patent No. 1,277,959 suggests an apparatus able to increase or to depress the excitability of the neural networks, according to the schemes scientifically defined of “sensitisation” or “habituation” (Kandel E R Eds Kandel E R, Schwartz J H and Jessell T M. Elsevier NY. 1991), by the prolonged application of suitable micro-lengthening sequences of the muscular fibres. Stimulus applied, and thus the controlled variable, in this apparatus is clearly only length, the variation of which constitute the sensorial stimulus read from the muscle length sensors.

Forces necessary to vary their length for millimetre fractions are very modest, since said tissues have a high yield.

Structure of the apparatus described in said Italian Patent is thus conceived to generate little motions in soft tissues, the amplitude (excursions) of which is the controlled variable.

Further, small forces applied to the cutaneous surface, destined to propagate micro length variations of the muscle constitute a succession of tactile stimulus for the cutaneous receptors.

It is known from the scientific literature that said cutaneous stimulus invariably involves an anaesthesia (loss of informative flow from the Nervous System) of the subjected to the treatment region (O'Mara S, Rowe M J, Tarvin R P, J Neurophysiol 59: 607-22, 1988), thus making it impossible to the patient the correct positioning of the limb. Since said apparatus requires the articular immobilisation in the muscular region subjected to the treatment, it requires a continuous and careful surveillance of a skilled operator, thus being different from the operation mode of other rehabilitative instruments such as electrostimulators, ultrasound generators, magnetic field generators, etc.

Among the other known apparatuses, none of them has the object of producing a neural conditioning paradigm, with or without minimisation of the co-operative role of the patient. Furthermore, none of the other apparatuses gives a response to the requisites necessary to produce a neural conditioning paradigm with, or without, minimisation of the co-operative role of the patient.

Some of the solutions of the prior art are not conceived to develop a frequency included in the range between 50 and 160 Hz, and based on a band of 80-120 Hz, in this range the Central Nervous System is particularly responsive to applied sensorial stimulus (Sitting A C, Denier van der Gon J J, Gielen C C Exp Brain Res 67: 33-40; 1987. Rollnick J D, Siggelkow S, Schubert M, Schneider U, Dengler R. Muscle Nerve 24: 112-115; 2001)). Particularly, U.S. Pat. No 3,984,708 provides an operative frequency of 50-60 Hz, U.S. Pat. No. 5,519,292 an operative frequency of 1-10 Hz, while U.S. Pat. No. 5,437,608 provides only a rotative massage.

In other documents, solutions are described that are not conceived to produce periodic, continuous and continuously modulate or that can be modulated forces, according to the wishes of the operator. Particularly, the solution according to the Italian Patent provides only impulsive forces, the U.S. Pat. No. 5,085,535 provides only impulsive forces, and U.S. Pat. No. 5,437,608 provides only a rotative massage.

Other solutions U.S. Pat. Nos. 4,895,149, 5,279,284, 5,113,852, 5,437,608, 5,519,292, 5,113,852 are conceived to have two parallel force applicators. Said arrangement produces two mechanical wave sequences, the propagation of which is destined to meet each other and to sum each other. Consequently, it will be obtained the production of a single wave having a frequency probably higher to 120-140-170 Hz, and in any case unavoidable from the operator action, and the efficiency of which will be verified only by the presence or absence of the wished results.

In some cases, instruments are explicitly conceived to produce forces having a very small amplitude, not destined to be transmitted up to osteo-articular tissues passing through soft tissues such as muscles, or, in others, the problem of the propagation of the force is not taken into consideration.

For example, it is true in the solutions described in the Italian patent No 1,277,959, aiming to impose micro-variations of the muscular fibre length, and in U.S. Pat. Nos. 3,984,708, 4,895,149, 5,101,810, 5,437,608, 5,519,292, 5,780,958, 5,611,771.

Furthermore, in some solutions provided and described in the above mentioned prior art documents, it is not faced up the problem relevant to the delivering of a force signal sufficiently broad to reach at least one of the articulations controlled by the muscle on which the apparatus is applied and, from this articulation, extending up to the adjacent bone, tendinous, ligament and soft tissues.

Moreover, said solutions are not conceived to be provided with stands able to be freely oriented in the space. Consequently, their use is completely limited, or even impossible, for most of the muscular areas involved.

This is, for example, the case of the solutions described in U.S. Pat. Nos. 3,364,921, 3,984,708, 4,549,535, 4,895,149, 5,085,207, 5,101,810, 5,113,852, 5,279,284, 5,361,437, 5,437,608, 5,519,292, 5,611,771, 5,780,958.

In some cases, the solution suggested is such to cover the body sector on which the stimulus is applied, and it is impossible for the operator to make a suitable control of the positioning of the apparatus, as well as of the propagation of the stimulus to the muscular, tendinous and osteo-articular territory.

In other cases, the solutions are not conceived to provide a suitable constraining reaction.

Still, some of the solutions described in the above mentioned patents are realised to be hand-held all along the duration f the application. In this case, it cannot be guaranteed the stability of the application and the operator should make a double work: to control the patient and to try to keep the same position for a time not lower than 5 minutes. Furthermore, the same operator should provide the constraining reaction necessary to the proper transfer of energy from the apparatus to the patient.

Finally, some of the solutions described in the above are conceived to work on a single and specific muscular territory.

Main object of the present invention is that of providing a method, and an apparatus, able to make three different, contemporaneous and synergetic actions.

Particularly, the first action consists in modifying the mechanical properties of the soft tissues, transforming them into high-pass filter able to isometrically transmit (i.e. without length variations) the force signal up to the nervous sensors present in the articular muscle groups. The second action consists in the simple and fast use of an alpha-conditioning paradigm able to induce in the patient subjected to its action a stimulation of its neuro-motive capability. Said action is based on a powerful increment of the managing of the informative flow about the inner condition relevant to the outer one (proprioceptive sensibility based on isometric mechanoreceptor), from the above descending:
an optimisation of the capability of the neuromuscular system to automatically oppose to the application of outer perturbation, increasing the muscular tone in case of hypotonia, or reducing the same in case of hypertonia deriving from neurological lesions in pyramidal site;
an optimisation of the energetic balance for the motion execution thanks to a stimulation of the proprioceptive control.

From the above:
it allows a reduction of the energetic expense and thus a drastic reduction of the sensibility to the muscular fatigue;
it allows to increase working loads;
it allows to obtain a reduction of the recovery time during the repeated physical exercises;
it allows to have an increase of the muscular co-ordination;
it allows an enlargement of the cerebral areas destined to the analysis of the sensitive signal useful for the motive co-ordination.

In case of tactile sensitive deficit due to neurological damages, determines a strong recovery of the sensitivity above the subjected to the treatment muscular territory.

Finally, at the same time, the method, and the apparatus, according to the invention induces in the patient an illusory perception of his articular positioning. Said perception produces in the subjected to the treatment muscle-articular territory both an involuntary contraction surplus, destined to further stiffen the tissues (exaltation of the high-pass features necessary to the propagation of the mechanical stimulus), and a maintaining of the position partially independent from the patient will. In this way, it is possible to minimise the need of co-operation from the patient, making it possible the use of the apparatus on each kind of patient (from an age of 4-5 years. Up to 90-95 years, on neurologic patients affected by ictus and consequently having minimum motion control features).

It is therefore specific object of the present invention a method for improving neuromuscular performances, characterised in that it is based on an Alpha-conditioning technique, carried out by a suitable stimulus of the nervous sensors during the voluntary muscular contraction, in that it transforms the mechanical properties of the soft tissues, developing a high-pass mechanical filter, in that it delivers a controlled and that can be modulated mechanical force signal destined to be read by force isometric nervous receptors, and in that it creates an illusory perception of the articular positioning.

Preferably, according to the invention, forces are applied having a time variable run in function of the application, having controllable intensity, prolonged times, fixed frequency, on force isometric nervous receptors, situated within deep tissues.

Furthermore, according to the invention, said forces must have such an entity to be read by the receptors.

In a preferred embodiment of the method according to the invention,
patient must be put in a comfortable position, and said position must be maintained for at least 10 consecutive minutes;
therapist must position the apparatus in such a way that the longitudinal axis of the transducer makes an angle as more as possible close to 90° with the cutaneous plane of the patient;
therapist must ask the patient the voluntary contraction of the territory to be subjected to the treatment, the level of said contraction must be such to be maintained for at least 10 consecutive minutes;
the operator must push the applicative part of the transducer against the patient tissues, thus obtaining a compression of the underlying tissues;
compression must be such that:
   tissues immediately surrounding the compression point are lengthened, up to making them reaching their upper stretching level for their elastic limit, so as to minimise the extensibility of the same tissues;
does not block the hematic flow, and thus does not avascularize the subjected to the treatment area.

Preferably, according to the invention, validity of the compression will be evaluated by the same operator both visually (inspective examination) and by palpation.

Furthermore, according to the invention, the apparatus, during the application of the forces, thanks to its conformation, will further compress said tissues and will bring the extensibility to still lower values, thus guaranteeing a suitable propagation of the force waves, said further compression, not having a continuous but a periodic amplitude, will allow to maintain a suitable and sufficient hematic microflow to feed the tissues under compression.

Still according to the invention, the same compression will be repeated in the following applications by reading of the values revealed by a force sensor (provided in the applicator) and/or deformation sensor (provided in the suspensions).

Furthermore, according to the invention, the compression will have to be carried out while the patient keeps the musculature to be subjected to the treatment in an isometric contraction (thus creating the treatment conditions).

The operator will have to behave differently in case subjects having strong force or fatigue resistance deficit. Said patients will obtain highly satisfying results producing an intermittent contraction.

Further, the operator will have to behave differently in case of patients having relevant spastic paralysis, so as not to allow any voluntary control of the same musculature. Said patients can be subjected to the treatment without voluntary contraction. In this way a strong and persistent muscular relaxation will be obtained, without motion control recovery.

Always according to the invention, intensity of the force to be applied during the treatment will be adjusted on the basis of two criteria:
information obtained from the patient;
palpation of the tissues.

The patient will have to inform the operator of the extension of the force propagation, indicating in which body territory he feels the mechanical vibration arriving.

The operator will have to verify the propagation by palpation.

Mechanical vibration will have to invade and to be clearly perceived in two, an at least one of the articulations controlled by the muscles subjected to the treatment.

Force intensity applied, when invading the articulations, will have not to generate pain.

Treatment to reach its full effect will have to be made for three following days, with at least three applications each day, each application lasting 8 minutes. Each application will have to be spaced of at least 15-30 seconds from the previous one.

Very modest effects, and persisting for no more than 7 days, can be obtained even with a single applicative session lasting 10 minutes.

The invention further relates to an apparatus for improving the neuromuscular performances, characterised in that it comprises at least a transducer, a transducer fixing system, and a control panel, said transducer having to support a static load adequate to transform biological tissues into a high-pass filter, imposing and sustaining a dynamic additive load on the muscular groups to be subjected to the treatment, thus producing a force signal able to propagate within the tissues, and being able to transmit said force signal to the patient; said control panel having to deliver an electric signal characterised by a pre-established frequency, being it possible to modulate the amplitude of said frequency (to reach the deep muscle and its articulation).

Particularly, said apparatus for improving the neuromuscular performances comprises a control panel comprised of a signal generator, an amplifier, a remote or resident control system, a timer, an intensity adjustment system, a system for evaluating the compression exerted, a transducer provided with a suspensions system, and an applicator group, and a support apparatus provided with adjustment for coarse and precision positioning, and constraining reaction.

Preferably, said apparatus provides a true transmission of the signal without distortion.

More specifically, signal generator of the control panel of the apparatus according to the invention must produce a continuous and periodic signal (preferably a sinusoidal signal, but also, as alternative, a sawtooth, triangular, trapezoidal, etc. signal), continuously controlled, modulated and that can be modulated in amplitude, the peak-to-peak values of which can be pre-set, or modified by the operator.

Particularly, peak-to-peak value will have to pass from the zero value and have its intermediate value in correspondence of the zero value.

Further, said signal must be continuous, periodicity being possibly at a pre-set frequency the basic harmonic of which will have to be at a single frequency to be chosen within a possible range included between 50 and 160 Hz, preferably between 70 and 140 Hz, still more preferably between 80 and 120 Hz, or characterised by a single harmonic having a value between 50 and 160 Hz, preferably between 70 and 140 Hz, still more preferably between 80 and 120 Hz.

Always according to the invention, said amplifier must not distort the signal in such a way to invalidate the features of the generator, must not introduce a bias, must guarantee the fixed frequency, must allow a suitable amplitude modularity of the signal by the operator, must provide the energy sufficient to generate forces between 0.5 and 100 Newton.

Furthermore, according to the invention, said transducer can comprise a winding, having a length higher than the length of a central cylindrical magnet, said magnet providing an inner hole and being connected, at one end, with a not-magnetic cylinder, said not-magnetic cylinder being provided with a through hole.

Further, according to the invention, two suspensions are provided, fixed by spacers to a container, said suspensions guaranteeing a rigidity between 1 and 30 N/m.

Always according to the invention, forces will have to be included between 2 and 100 Newton.

Still according to the invention, a deformation sensor can be applied on one of said suspensions.

Furthermore, according to the invention, it can be provided a not magnetic material cylinder, provided with two pins to be fixed to the support.

Always according to the invention, said applicators of said transducer provide a portion that must be fixed to the magnet and one or more portions that will be in touch with the patient, said part can be inserted within said hole.

Further, according to the invention, force sensors could be applied on the top of said applicator.

Still according to the invention, said support can provide a multiple articulated joint and arm system to reach each point on the patient body (it is possible to provide 9 freedom degrees for the whole structure), and from this point to exert the force according to each direction.

Furthermore, said transducer and the applicators have such a physical configuration to allow a full visibility of the cutaneous territory immediately close to the applicator, said stand having a configuration suitable to guarantee a suitable and graduated compression of the tissues to be subjected to the treatment, said stand having a configuration suitable to constantly sustain the static load produced by the compression of the tissues, and said stand having a configuration suitable to guarantee the force transmission from the transducer to the tissues to be subjected to the treatment.

The present invention will be now described, for illustrative but not limitative purposes, according to its preferred embodiments, with particular reference to the figures of the enclosed drawings, wherein:

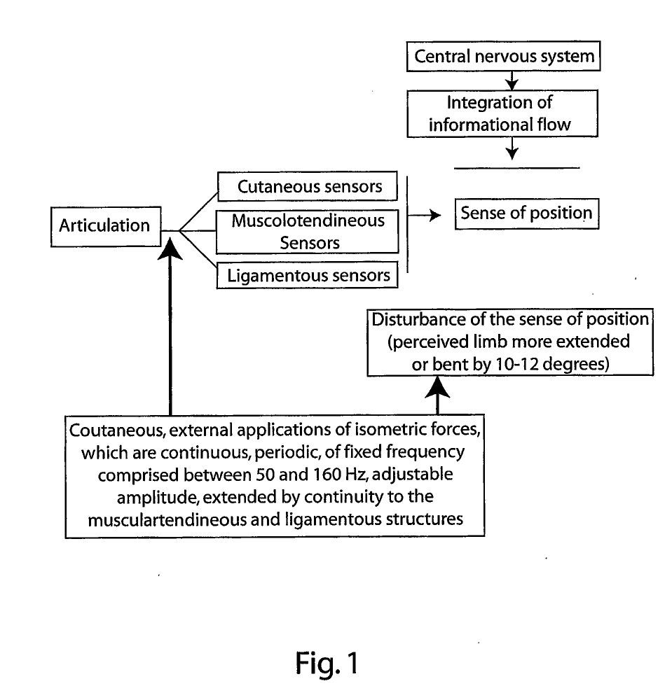

FIG. 1 shows a block diagram relevant to the articular perception;

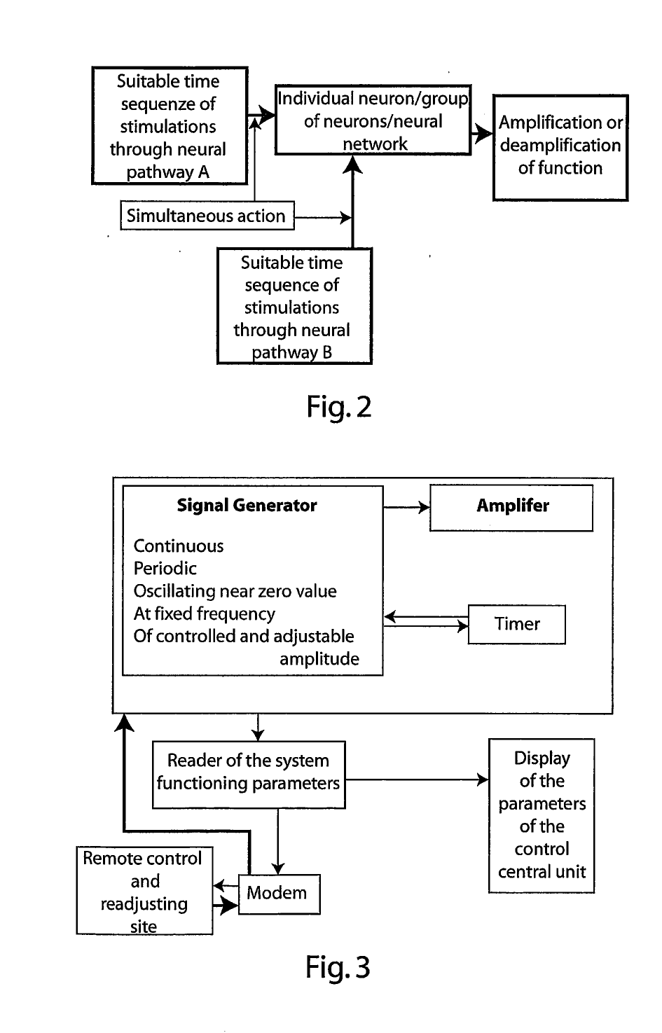

FIG. 2 shows a block diagram of a paradigm named “alpha-conditioning”;

FIG. 3 is a scheme of the electronic components of an embodiment of the apparatus according to the invention;

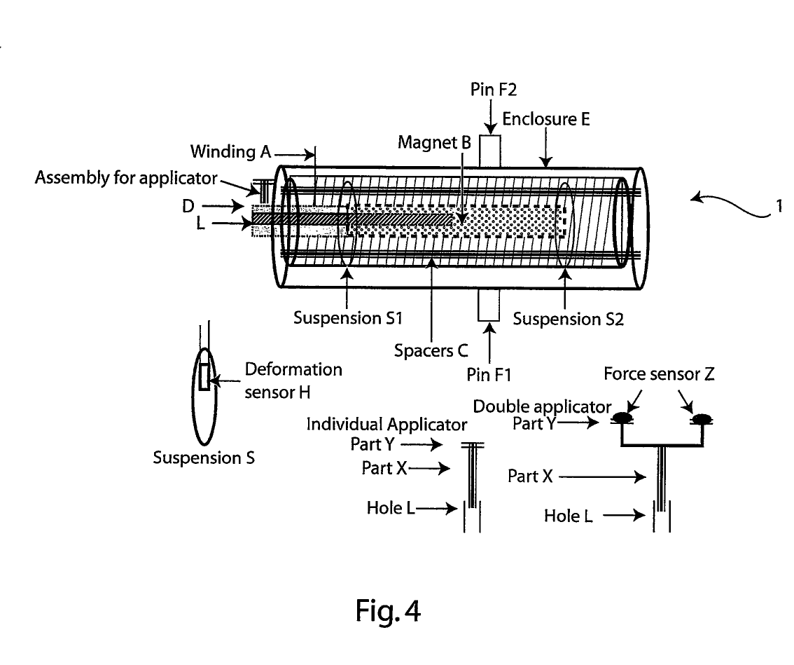

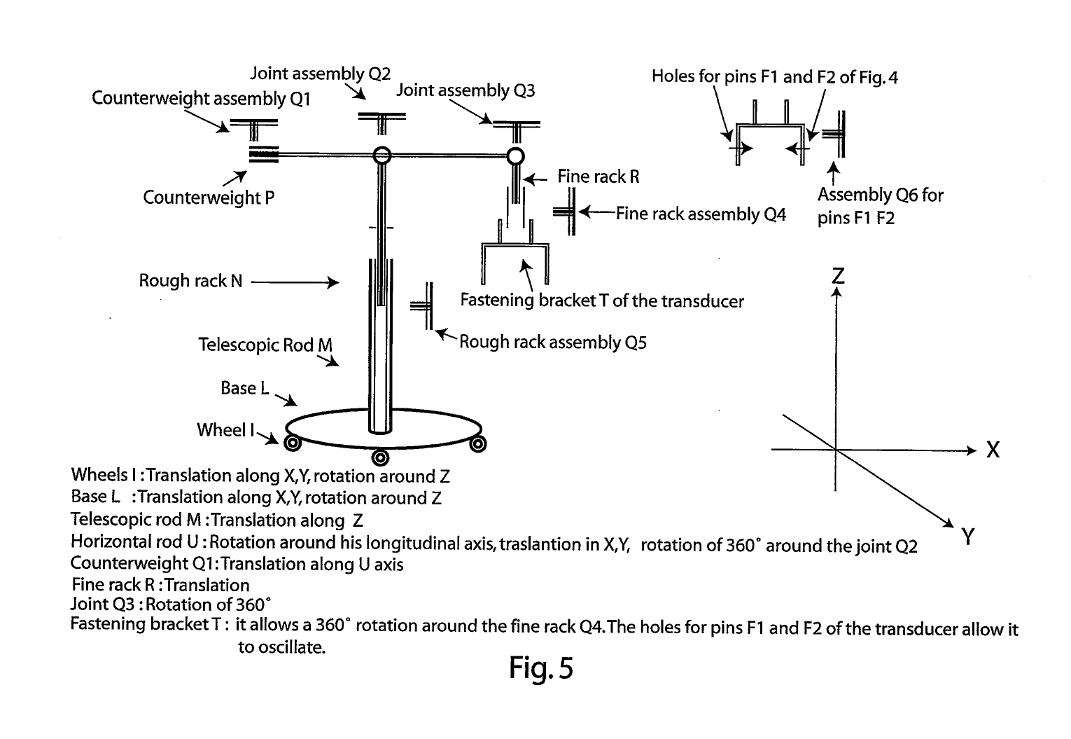

FIG. 4 schematically shows an embodiment of a transducer system;

FIG. 5 schematically shows an embodiment of a support of the apparatus according to the invention;

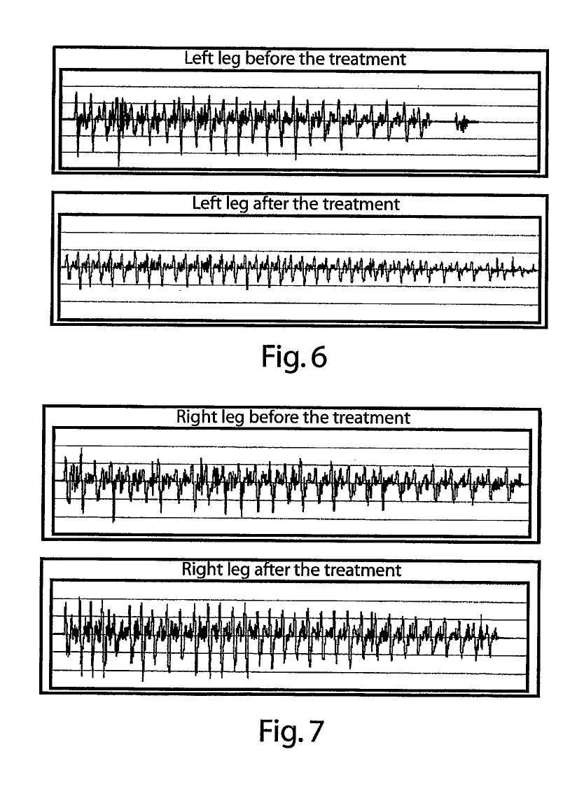

FIG. 6 shows a graph relevant to a control and to a test on the left leg; and

FIG. 7 shows a graph relevant to a control and to a test on the right leg.

Making now reference to the figures of the enclosed drawings, it is described an embodiment of the apparatus according to the invention that, in this solution, provides a control panel (see particularly FIG. 3), comprised of a signal generator, an amplifier, a remote or resident control system, a timer, an intensity adjustment system and a system for evaluating the compression exerted.

The apparatus further provides a transducer (see FIG. 4) provided with a suspensions system, and an applicator group.

In FIG. 5 it is further shown an embodiment of a support apparatus provided with adjustment for coarse and precision positioning, and constraining reactions.

Coming now to describe in greater detail the various components of the apparatus according to the invention, in the control panel, the signal generator must produce a continuous and periodic signal (preferably a sinusoidal signal, but also, as alternative, a sawtooth, triangular, trapezoidal, etc. signal), continuously controlled, modulated and that can be modulated in amplitude, the peak-to-peak values of which can be pre-set, or modified by the operator.

The peak-to-peak value will have to pass from the zero value and have its intermediate value in correspondence of the zero value. Said signal must be continuous, periodicity being possibly at a pre-set frequency the basic harmonic of which will have to be at a single frequency to be chosen within a possible range included between 50 and 160 Hz, preferably between 70 and 140 Hz, still more preferably between 80 and 120 Hz, or characterised by a single harmonic having a value between 50 and 160 Hz, preferably between 70 and 140 Hz, still more preferably between 80 and 120 Hz.

The amplifier must not distort the signal in such a way to invalidate the features of the generator, must not introduce a bias, must guarantee the fixed frequency, must allow a suitable amplitude modularity of the signal by the operator, must provide the energy sufficient to generate forces between 0.5 and 100 Newton.

In any case, forces included between 3 and 5 Newton are an acceptable range for applications on children up to 10 years of age, and in the masticator apparatus of the adults.

Instead, values included between 5 and 60 Newton are a valid range to work on adults and athletes.

As to the remote or resident control system, proper operation of the control panel, working time, its modes, will be evaluated (e.g. for the technical assistance) by a working parameter revealing system and a modem to be used through the Internet, or more generally, through the phone. Reading of the same parameters can also be made locally, by indicators placed on the same control panel.

Timer is destined to set the application time of the apparatus on the patient, allowing automatic switching on and switching off.

By the intensity adjustment system, while frequency can be fixed, maximum intensity applied can be finely adjusted by the operator in function of the needing (territory subjected to the treatment, features of the patient: height, weight, fat mass, etc.). Force developed will be read on a display.

As already said, it is provided an evaluation system of the compression exerted by the transducer.

In fact, soft tissues must be placed under load by the transducer: load will be read by a load cell or other deformation sensor and the signal will be sent to an amplification stadium and read on a suitable display.

As to the transducer, it has two roles: a passive role and an active role.

Passive role: it must be able to compress the cutaneous and subcutaneous soft tissues (adep layers) so as to minimise the dampening action of the applied forces. Said compression will determine a load (preload) additional with respect to the load that the transducer will have to sustain when developing the force during its action. Said preload will vary between 100 g and 4 Kg, on the basis of the kind of underlying soft tissues, of the extension of the territory to be subjected to the treatment (e.g. child or adult) etc.

Active role: it must be able to true transduce during the time and in amplitude the periodic force electric signal, continuously controlled, said amplitude being modulated and that can be modulated according to the operator intentions. It must further allow the application of said force to the underlying tissues guaranteeing an intensity suitable to the propagation at least up to an articulation and to its adjacent tissues.

To reach said objects, it is indispensable:
to guarantee a suitable rigidity to the transducer structure destined to transmit the force signal. Said rigidity will have to guarantee both the maintenance of the proper attitude of the components of the transducer subjected to the preload and, when the transducer is active, a “not deformation” of the force signal.
to realise a structure able to always and in any case transform the electric signal into mechanical signal that is constantly controlled by the control panel.
to true and without distortions (friction, not foreseeable constraining reactions, etc.) translate the signal arriving to the transducer, avoiding to invalidate the features set for the generator and the amplifier.

Making specific reference to FIG. 4, said transducer 1 provides a winding A, having a length higher than the length of the central cylindrical magnet B. Said arrangement is necessary to prevent that possible displacement of the magnet, for example during the compression of the soft tissues, reduces the number of turns of the winding A surrounding the cylindrical magnet B and at the same time reduces the portion of the magnet exposed to the action of the current passing through the turns.

Magnet B has an inner hole (L). Magnet B will be connected, at one end, with a not-magnetic cylinder M, said not-magnetic cylinder being crossed by the same hole L. Said magnet further provides two suspensions (S1, S2), having a known rigidity, fixed by spacers C to the container F. Role of the suspensions S1 and S2 is to guarantee a rigidity between 1 and 30 N/m, in function of the power that it is set to have at disposal. It must be noted that higher is the power, higher is the capability of intervention of the system on body portions characterised by large masses, both fat and muscular masses.

Generally speaking, the following rigidity and force pair values can be indicated: 1-2.5 N/muscles for forces included in the range between 2 and 6 Newton; 2.5-3.5 N/muscles for forces included in the range between 6 and 9 Newton; 3.5-15 N/ muscles for forces included in the range between 9 and 20 Newton; 15-20 N/muscles for forces included in the range between 20 and 50 Newton; 20-30 N/ muscles for forces included in the range between 50 and 100 Newton.

Rigidity values included between 1 N/mm and 3.5 N/mm can be considered suitable for applications on muscular-articular masticator territory for adults and children and on all the muscular articular territory of children up to 10 years of age. Values included within the range between 3 N/mm and 30 N/mm are suitable to treat each muscular articular territory of adults and children.

Forces will have to be included in the range between 2 and 100 Newton. Forces between 2 and 9 Newton are an acceptable range to work on children up to 10 years of age, and on the masticator apparatus of everybody. Values included between 9 and 100 Newton, more preferably between 12 and 70 Newton, still more preferably between 10 and 50 Newton, are a valid range to work on adults and athletes.

Said suspensions will have the task to maintain magnet B at the centre of the turns, avoiding that loads not coincident with the longitudinal axis of magnet B, and thus with the force vector acting on the same, can modify the attitude of the transducer, damaging or making it temporarily inefficient. A deformation sensor H can be applied on one of said suspensions, so as to inform the operator if the apparatus compresses the soft tissues of the patient. Said components A, B, C, D, H, S1, S2 are included within a not magnetic material cylinder E, provided with two pins F1 and F2, to be fixed to the support G.

Furthermore, applicators are provided on the transducer 1, to transmit forces generated by the transducer directly on the patient.

Said applicators will have different shape and dimensions, suitable to treat different muscles in anthropometrically different patients. Generally speaking (see FIG. 4) it will be possible top distinguish in each applicator a part X destined to be fixed to magnet B and one or more parts Y destined to be in touch with the patient. Part X can be inserted within hole L and suitably fixed.

Applicators will have to be properly fixed in such a way to resist in the proper position during the production of forces and will have to be easily removed and replaced by the operator. On the top of said applicator force sensors (Z) could be applied, giving information about the kind of load applied on the soft tissues during the positioning of the apparatus and about the force applied by the transducer during its operation.

Making now reference to FIG. 5, it is shown a support apparatus, generically indicated by reference number 2, having the task of guaranteeing the best orientation of the transducer and of its applicative part to the muscular group, a suitable compression of the soft tissues and a suitable constraining reaction.

It can be provided a multiple articulated joint and arm system to reach each point on the patient body (it is possible to provide 9 freedom degrees for the whole structure), and from this point to exert the force according to each direction.

The precision of the tissue compression necessary to allow the best propagation of the mechanical energy wave continuous sequence can be obtained by two racks having different stroke pitches. The first one will allow a coarse approach (pitches of 5 mm, about 50 cm of total stroke). The second one will have micrometric features, with pitches of 0.1-0.5 mm and total stroke of 5-10 cm). Strokes of the two racks will be blocked by screws or by blocks perpendicular to the stroke direction, or provided or assembled, or by other devices guaranteeing both resistance to the vibrations and an adequate constraining reaction.

Block of racks, weight of support apparatus, block of articulated joint points of the support apparatus will have to guarantee the necessary constraining reactions.

The constraining reactions produced by the instrument will have not to lower than 2 Kgf for instruments delivering 2-6 Newton; will have not to lower than 3 Kgf for instruments delivering 6-9 Newton; will have not to lower than 4 Kgf for instruments delivering 9-20 Newton; will have not to lower than 5 Kgf for instruments delivering 20-25 Newton; will have not to lower than 6 Kgf for instruments delivering 50-100 Newton.

As it can be clearly noted from the above, method and apparatus according to the invention allow to generate a continuous succession of periodic force waves, continuously controlled and modulated in amplitude. Said waves must have such features to be propagated through soft tissues such as cutis, adeps and muscles up to tendons and osteo-articular tissues, and from here up to other tendons inserted on the same osteo-articular segments.

Said applied force variations are destined to be read by the nervous sensors sensitive to the isometric forces and to their time derivative.

The apparatus will allow to apply said continuous succession of waves on muscular groups or on single muscles, but the apparatus according to the invention is conceived in such a way to generate a mechanical signal (particularly characterised by isometry and suitable amplitude and frequency parameters), so as to reach the articular, osseous, ligamentous, and thus adjacent tendinous tissues, and from here, to invade other muscular groups involved in the control of the same articulation. In this way, nervous receptors involved by the stimulus will be the mechanical, isometric one, present also in the above mentioned structures.

As noted in the above, method, and apparatus, according to the invention, is conceived in such a way to act on the mechanical features of the soft tissues, and on two propertied of the nervous system:
a graduated compression of the soft tissues transforms the same into high pass filters, without arriving to occlude the vas during the applicative time (minimum value 8 minutes);
possibility of modifying the neural networks by adequate stimulus. In this case, the adequate stimulus is the application of the isometric mechanical forces able to be read by isometric force nervous receptors (Bear M F and Malenka R C Curr Opin Neurobiol 4: 389-399. 1994; Byrne J H Physiol Rev 67: 329-439. 1987; Kandel E R Eds Kandel E R, Schwartz J H and Jessell T M. Elsevier NY. pp 1009-1031. 1991; Mendell L M Physiol Rev 64: 260-324. 1984);
capability by some suitable stimulus to modify the perception of the realty by the Central Nervous System, giving phenomenon that nowadays are the basis of the creation of virtual realty (Rizzo A A, Buckwalter J G Stud Health Technol Inform 44: 123-145; 1997)). In the specific case, features of the mechanical stimulus produced by the apparatus will be such to produce both a modification of the neural networks, and a stimulation of the articular perception, and thus of the position sense of the articular segment subjected to treatment (Roll J P, Vedel J P Exp Brain Res 47: 177-90; 1982; Sitting A C, Denier van der Gon J J, Gielen C C Exp Brain Res 67: 33-40; 1987; Verschueren S M, Cordo P J, Swinnen SP J Neurophysiol 79: 2265-76; 1998).

The apparatus according to the invention is further conceived in such a way to transform the soft tissues on which it acts in a high-pass filter indispensable to the transmission of force signals.

The apparatus according to the invention is further conceived to produce a conditioning paradigm defined as “alpha-conditioning” (Kandel E R Eds Kandel E R, Schwartz J H and Jessell T M. Elsevier NY. pp 1009-1031. 1991 and to develop a picture of virtual frame.

Alpha conditioning is based on the time association of two adequate stimulus contemporaneously presented to the Central Nervous System.

The apparatus suggested exploits the theory of the alpha conditioning and thus provides the time association of two qualitative and quantitative stimulus adequate to the achievement of the effects, the first one of which is generated by the same apparatus.

The latter is able to activate a large network of peripheral sensors, pertinent to the same articulation and characterised by their specific sensibility to isometric force variations. Further, it allows to associate in the space (same neural circuits) and in the time (association is contemporaneous) said stimulus to the activation of the neural networks controlling the motility of the same articular area (second stimulus, see the following specification).

In the multitude of nervous sensors present in the muscular-articular regions, the apparatus is destined to activate those sensible to the isometric force and to its derivative. Said activation will occur by propagation, through the muscular, tendinous, ligamentous, connective, articular and osseous tissues pertinent to at least one articulation, of a continuous succession of waves of periodic forces continuously controlled and modulated in their amplitude. Propagation of the waves will occur realising at least one physical continuity point between the apparatus and the cutis of the patient.

Apparatus will have to be adequate to compress underlying soft tissues, so as to minimise their dampening action of the amplitude of the force applied. In fact, it must be noted that high compliance of cutis, adeps, and muscular bands, even if partially contracted, tends to transform their force applied into simple movement.

Second stimulus is provided by the subject and sustained by the apparatus.

In fact, the patient will have to isometrically contract the muscular territory (thus without generating any movement), the performances of which must be increased and within which the mechanical energy waves developed by the apparatus travel. The patient will have to maintain the isometric contraction for the whole application time of the first stimulus (from 5 to 20 minutes). It must be noted that each voluntary and static action (i.e. involving maintaining a position, uniformly prolonged in the time) (for our Central Nervous System a prolonged time is a time longer than 15 seconds), generating a fast loss of the control due to the reduction of the informative signals destined to decay during the time if no variation occurs.

Furthermore, in the specific case, mechanical stimulus applied to the cutis in a repetitive way rapidly produce a strong and deep cutaneous anaesthesia condition (Sitting A C, Denier van der Gon J J, Gielen C C Exp Brain Res 67: 33-40; 1987)).

Anaesthesised cutaneous area extends on a surface having a ray of about 2-10 cm, having its centre on stimulus application point, the anaesthesia condition disappearing after about 15-30 minutes from the end of the treatment.

Said anaesthesia, associated both to the decaying of the information destined to the Central Nervous System, about the position maintained by the limb, and to the anaesthetising action of the repetitive tactile stimulus delivered from the apparatus, always generates in the patient the need of moving the limb subjected to treatment to come back to a normal perception condition (moving it introduces dynamic components erasing the sensibility loss).

Movement of the patient produces a contact loss with the apparatus, with the consequent achievement of the wished effects and/or a damage of the same apparatus that can be seriously damaged from the sudden forces imposed by the patient movement.

The apparatus according to the invention prevents said situation, realising an illusory perception of iperextension or iperflexion of the articulation. The patient automatically adequates the contractile status of the responsible musculature to the virtual realty thus elaborated. Said illusory position perception is obtained thanks to the capability of the apparatus to invade the articulations, tissues and osseous segments adjacent to the same with the continuous succession of isometric periodic force waves continuously modulated and controlled in their amplitude. It is in fact known that the discontinuous stimulus in the time of an articular territory produces an illusory perception of motion (useless and harmfulness of using forces not continuous during the time in this case). Instead, on the contrary, a stimulus without discontinuity increases the articular positioning sense of about +10-12° with respect to the real situation (Sitting A C, Denier van der Gon J J, Gielen C C Exp Brain Res 67: 33-40; 1987)).

Further, within the range 80-120 Hz of operation of the apparatus, it is particularly efficient on the Central Nervous System, for reasons still not clear. (Sitting A C, Denier van der Gon J J, Gielen C C Exp Brain Res 67: 33-40; 1987. Rollnick J D, Siggelkow S, Schubert M, Schneider U, Dengler R. Muscle Nerve 24: 112-115; 2001).

In this way, the apparatus not only produces cutaneous and deep anaesthesia, but also induces in the subject an illusory perception of articular position making it easy or even spontaneous maintaining the required position.

Induction of said phenomenon is due to the specific features of the apparatus according to the invention:

Structure: able to compress cutaneous and subcutaneous soft tissues; transforming tissues into high-pass filters and establishing a mechanical rigidity continuity between control panel and force isometric mechanical nervous sensors. Able to orient according to each direction the generated force vector, able to provide the necessary constraining reaction, and able to maintain the wished position without requiring the continuous presence of the operator.

Kind of signal provided generated: continuous succession of periodic forces, continuously modulated and controlled in their amplitude.

Signal amplitude: adequate to the propagation through soft and adjacent hard tissues. Adequate to invade at least one articulation close to the force signal application point.

Frequency: fixed to a value between 50 and 160 Hz, preferably between 80-120 Hz, or characterised by a single harmonic the value of which is included between 50 and 160 Hz, preferably between 70 and 140 Hz, still more preferably between 80 and 120 Hz.

Said features, as a whole, allow to exploit the importance of the articular and isometric force sensors in determining the perception of a virtual realty relevant to the articular position, promoting both the production of a mechanical high-pass filter, and the maintenance of the position required by the patient.

In the following some peculiar features of the method and apparatus according to the invention will be indicated.

Necessary conditions to obtain the wished effect are that the patient puts himself and the limb to be subjected to treatment in an position suitable to be maintained for a time between 5 and 30 minutes, preferably between 7 and 20 minutes, and still more preferably between 10 and 15 minutes.

The patient must put the muscle or the muscular groups to be subjected to treatment in a clear contraction condition: thus it must produce a local muscular contraction, so as to generate movement. In case of pyramidal spastic paralyse, optimisation of the tone will be obtained also without the voluntary contraction.

Apparatus must be put in touch with the cutaneous surface above the territory to be subjected to treatment, preferably, but not compulsorily, close to the muscular-tendinous joint.

The part of the apparatus destined to generate and propagate the suitable continuous succession of periodic isometric forces continuously modulated and controlled in their amplitude, must compress underlying soft tissues (cutis, subcutaneous, adeps, surface muscular fibres) in a suitable way to minimise the dampening phenomenon of the mechanical energy applied through a transformation of the force into simple movement. Said compression will have to contribute to guarantee the propagation of the isometric force variation through soft tissues such as cutis, adeps, muscles, up to tendons and osteo-articular tissues, and from here up to the other tendons inserted on the same osteo-articular segments.

Apparatus must be blocked in such a way to guarantee the transmission of the force wholly to the patient.

Force generated must be such not to cause articular pain.

The continuous sequence of waves of periodic isometric forces continuously modulated and controlled in their amplitude must be such to propagate and clearly invade the muscle(s) to be subjected to treatment, its tendons, at least one of the articulation on which muscular structures act, osseous tissues adjacent to the same articulation.

Said propagation must be verified by the operator.

Treatment must be comprised of at least two daily sessions, each session lasting at least 5 continuous minutes, two following sessions must be spaced of a short interval of at least 15 seconds. The treatment must be repeated for at least two days in order to have effect maintained for months. A single applicative day can be carried out if persistency of the effects must be lower than a week (effects lasting 4-6 days).

Therefore, apparatus must deliver a continuous, periodic force signal, continuously modulated and the amplitude of which can be modulated according to the intention of the operator, by a suitable system.

Periodicity will have to be at a pre-set frequency and its basic harmonic must have a single frequency to be chosen in a possible range between 50 and 160 Hz, preferably 70 and 140 Hz, still more preferably between 80 and 120 Hz, or characterised by a single harmonic the value of which is included in the range between 50 and 160 Hz, preferably 70 and 140 Hz, still more preferably between 80 and 120 Hz.

Further, the apparatus must deliver a force signal enough wide to reach at least one of the articulation controlled by the muscle on which the apparatus is applied and, from this articulation, extend up to adjacent osseous tissues, tendinous, ligamentous and soft tissues.

Signal delivered must oscillate about the value of zero Newton, so as to prevent that the apparatus imposes to the nervous receptors a “bias” due to a constant force. Said “bias” would be translated into a variation of the perception of the wished articular position.

Soft tissues must be compressed in such a way to minimise their compliance, without deforming or modify its force applicative capability.

Said compression of the soft tissues must be controlled by the therapist.

Mechanical energy must be transferred to the patient without being absorbed by the support structure of the apparatus in such a way to make null the propagation.

Further, apparatus must allow to reach each point in the space useful to treat a patient without requiring the continuous presence of the operator.

From that point, force vector will have to be oriented according to each direction and verso.

The same apparatus will have to be structured in such a way to be applicable on each body portion, making it adequate to operate on different territory, such as the masticator, muscles of the hand, surae quadriceps or triceps, perineal musculature and abdominal musculature, etc.

In the following, an example of application of the method according to the invention will be described for exemplificative, but not limitative purposes.

Results obtained during a test before the treatment with the apparatus and during a test after the treatment with an apparatus. Time between the treatment and the test was of 15 days. During this period, the subject had a completely sedentary life. Features of the treatment: 3 following days, 3 sessions for each day lasting 10 minutes each one, spaced of 1 minute of rest, on the Vasto Mediale, intermediate recto, rectofemouris of the right thigh (not dominated limb) muscles.

Test modes:

Patient seated, extension movement of a single leg subjected to a load of about 25% of the body weight of the subject.

Each leg carried out 3 following series separated by 10 minutes of rest. During each series the subject had to fully extend the leg (knee angle of 180°) and come back to the initial position (knee angle of 90°).

During each series, the subject had to carry out the maximum possible number of repetitions. Measured parameters: number of repetitions for each series, acceleration of the leg.

Muscle used to carry out the movement required: quadriceps muscle.

Muscle subjected to treatment: only the left quadriceps, interesting also the knee articulation.

Each graph shows the run of the acceleration (positive upward, negative downward).

Each group comprised of a single upward wave, followed by a wave downward, represents a single extension movement followed by the return to the rest position.

It must be noted (FIG. 6) that, after the treatment, the sole modifications are evident only on the leg (left) subjected to treatment.

The plot shows on the side subjected to treatment a very clear reduction of amplitude, even remaining unmodified the amplitude of the spanned articular angle.

Peak relevant to the descent is reduced: it appears a bigger control of the leg return, thus a better proprioceptive control and better motion coordination.

Time between consecutive exercises reduces for the lower energetic expense: reduction of the recovery time.

Lower energetic expense will make it possible to increase the working loads.

Apparatus will have to be structured in such a way to make it visible to the operator the area immediately surrounding the application point, so as to allow a visual and palpation examination of the proper application of the mechanical stimulus.

Summarising, the invention also concerns an apparatus to improve the neuromuscular performances, characterised in that it comprises at least a transducer, a stand and a control panel.

Said transducer and stand must realise an integrated system to make a high-pass tissue filter without damaging the microcirculation and creating isometric working conditions.

Said transducer must be able to sustain a static load suitable to transform biologic tissues into a high-pass filter. Said transducer must be able to truely transduce into a mechanical signal the electric signal generated by the control panel into mechanical signal without inducing distortions. Said transducer must be able to impose and sustain an additional dynamic load to muscular groups to be subjected to treatment. Said transducer must be oriented according to every direction and blocked in every point close to the patient. Said transducer must be able to apply the dynamic load producing a further compression of tissues, so as to limit the possible occlusion of the microcirculation to an absolutely limited time period. Said transducer must be able to produce an isometric force signal able to propagate into the tissues. Said transducer must be able to transmit said force signal to the patient without dampening it. Said transducer must be able to develop its mechanical energy according to a single direction. Said transducer must be provided with applicator adaptable to the dimensions of the muscular territory to be subjected to treatment and having suitable rigidity to true transmit the signal generated by the transducer to the biological tissue, without causing distortions. Said transducer must be oriented according to every direction.

Said stand must be able to orient the transducer according to every direction. Said stand must be able to immobilise the transducer in every position. Said stand must be able to compress in a controlled, adjustable, continuous and not discrete manner soft tissues (cutis, subcutis and muscle). Said stand must be able to sustain said static load. Said stand must be able to sustain the dynamic load imposed by the transducer. Said stand must be able to develop a constraining reaction suitable to absolutely make immobile the transducer. Said stand must be able to dampen and to block the vibrations transmitted by the transducer in order to avoid both mechanical energy wave reflection phenomenon on the same transducer and from the latter to the biological tissues (loss of faithfulness), both resonance phenomenon, potentially harmful for the patient and for the instrument.

Said transducer and stand must realise an integrated system to make a mechanical continuum with biological tissues up to the isometric force nervous sensors.

Said control panel must deliver an electric signal that can be transduced by the transducer. Said signal will have to be characterised by a pre-set frequency the amplitude of which can be modulated (to reach muscles in deepness and the articulations). Said signal will not have to contain subharmonic to modify said pre-set frequency. Signal delivered will have to symmetrically extend above and under the zero value. Said panel control will not have to introduce a bias overlapped to the wished signal (not to modify the virtual realty signal).

Experimental demonstration of the selective activation of isometric force sensors and of the activation of the neuromuscular fusi by the inventive solution

The invention provides an instrument, which is new under a technical and neurophisiological test point of view. The new instrument is able to activate only the isometric force sensors, without involving the neuromuscular fusi.

Description of the results preliminar to the scientific publication obtained with the Human Physiology Institute of Perugia University, Medicine and Surgery Faculty, managed by Prof. V. E. Pettirossi.

Three subjects have been subjected, one by one, on a rotative footboard within a dark room. Before switching off the lights and closing the room, each a target placed on the opposite wall has been indicated to each subject, and they have been asked to close their eyes.

Then, the light have been switched off and the patient, immobile, have been passively rotated with a movement of the footboard according to a clockwise rotation of 90°. Light have been switched on while the patient, always with its eyes closed, had to indicate the target position according to its orientation perception. Subjects used have been previously trained and the mistaken was constant.

Each subject showed an orientation mistaken with a left overestimation of the target. Subject 1: it individuated the target at 87 cm+3 from the real position. Subject 2: 110 cm+5. Subject 3: 98 cm+4. Four consecutive tests have been carried out for each subject.

Then, the muscular group of each subject, involved in the head right rotation has been twice subjected to treatment by the instrument, 10 minutes each time.

The test have been repeated and the orientation error almost annulled.

Subject 1: 2 cm+1. Subject 2: 4 cm+1.5. Subject 3: 2,5 cm+0.5. Effect was maintained for 10 days.

Interpretation
each muscle contains tenth of thousands of mechanical sensors. Sensors of head rotation muscles are used to evaluate the spatial orientation of the head with respect to the trunk.

Thus, they contribute to the spatial orientation of the subject and in suppression conditions of the visual information (dark room), their contribution is powerful. A class of sensors controls the muscle length (thus informs the Nervous System about the lengthening of the muscle), a second class controls the forces developed by the contraction (thus informs the Nervous System about the contraction of the muscle).

Mechanical stimulus of the length sensors of the right head rotative muscles (neuromuscular fusi) would have signalled a left head rotation (right muscles lengthened) and a consequent increase of the error when individuating the target. Viceversa, a reduction of the error (result obtained) involves a lack of stimulus of the neuromuscular fusi and a powerful stimulus of the isometric mechanical sensors able to reveal the parameters of a muscular contraction in progress. Said situation is equivalent to the perception of a deviation of the head rightward, thus obtaining a reduction or reset of the error when individuating the target.

As it was expected, isometric conditions imposed by the instrument have not activated the neuromuscular fusi, completely inactive in isometry, but have powerfully activated isometric mechanical sensors.

The present invention has been described for illustrative but not limitative purposes, according to its preferred embodiments, but it is to be understood that modifications and/or changes can be introduced by those skilled in the art without departing from the relevant scope as defined in the enclosed claims.

1. Method for improving neuromuscular performances, characterised in that it is based on an Alpha-conditioning technique, carried out by a suitable stimulus of the nervous sensors during the voluntary muscular contraction, in that it transforms the mechanical properties of the soft tissues, developing a high-pass mechanical filter, in that it delivers a controlled and that can be modulated mechanical force signal destined to be read by force isometric nervous receptors, and in that it creates an illusory perception of the articular positioning.

2. Method according to claim 1, characterised in that forces are applied having a time variable run in function of the application, having controllable intensity, prolonged times, fixed frequency, on force isometric nervous receptors, situated within deep tissues.

3. Method according to claim 1, characterised in that said forces are quantitatively readable by the nervous mechanical and isometric sensors in the surface and deep tissues immediately close to the signal delivery point.

4. Method according to claim 1, characterised in that said forces are quantitatively readable by the nervous mechanical and isometric sensors in the surface and deep tissues distant up to 180 cm from the signal delivery point.

5. Method according to claim 1, characterised in that said forces are able to invade at least two articulations close to the signal delivery point.

6. Method according to claim 1, characterised in that said forces are able to invade at least one articulation close to the signal delivery point.

7. Method according to claim 1, characterised in that said forces are able to induce in the patient receiving said signal an illusory or virtual perception of its articular position.

8. Method according to claim 1, characterised in that:
the patient must be put in a comfortable position, and said position must be maintained for at least 10 consecutive minutes;
the therapist must position the apparatus in such a way that the longitudinal axis of the transducer makes an angle as more as possible close to 90° with the cutaneous plane of the patient;
the therapist must ask the patient the voluntary contraction of the territory to be subjected to the treatment, the level of said contraction must be such to be maintained for at least 10 consecutive minutes;
the operator must push the applicative part of the transducer against the patient tissues, thus obtaining a compression of the underlying tissues;
the compression must be such that:
tissues immediately surrounding the compression point are lengthened, up to making them reaching their upper stretching level for their elastic limit, so as to minimise the extensibility of the same tissues;
does not block the hematic flow, and thus does not avascularize the subjected to the treatment area.

9. Method according to claim 1, characterised in that validity of the compression will be evaluated by the same operator both visually (inspective examination) and by palpation.

10. Method according to claim 1, characterised in that said static load can be in the range between 50 g and 4 Kg.

11. Method according to claim 1, characterised in that said static load can be in the range between 50 g and 2 Kg, for a pre-pubertal age.

12. Method according to claim 1, characterised in that said static load can be in the range between 100 g and 4 Kg, for a pubertal and adult age, in function of the muscular mass and of the subcutaneous adep layer.

13. Method according to claim 1, characterised in that the same compression can be repeated in the following applications by reading the values revealed by a force sensor (provided on the applicator) and/or a deformation sensor (provided on the suspensions).

14. Method according to claim 1, characterised in that the compression must be carried out while the patient keeps the musculatur to be treated in an isometric contraction (thus creating the treatment conditions).

15. Method according to claim 1, characterised in that during the application of the forces will further compress said tissues and will bring the extensibility to still lower values, thus guaranteeing a suitable propagation of the force waves, said further compression, not having a continuous but a periodic amplitude, thus allowing to maintain a suitable and sufficient hematic microflow to feed the tissues under compression, preventing pain and damaging of the tissues.

16. Method according to claim 1, characterised in that the delivered mechanical signal, reaching the mechanical, isometric nervous receptors, in the articulations is able to induce in the patient an illusory or virtual perception of articular position, said perception being such to induce a supplemental and involuntary rigidity of the muscular-articular structure subjected to treatment.

17. Method according to claim 1, characterised in that intensity of the force to be applied during the treatment is adjusted on the basis of the force wave diffusion until at least two articulations close to the mechanical signal application point.

18. Method according to claim 1, characterised in that intensity of the force to be applied during the treatment is adjusted on the basis of the force wave diffusion until at least two articulations close to the mechanical signal application point.

19. Method according to claim 1, characterised in that intensity of the force to be applied during the treatment will be adjusted on the basis of two criteria:
a) information obtained from the patient;
b) palpation of the tissues.

20. Method according to claim 1, characterised in that the treatment is made for three following days, with at least three applications each day, each application lasting 8 minutes, each application will have to be spaced of at least 15-30 seconds from the previous one.

21.-41. (canceled)
